(19) United States
(12) Patent Application Publication
(10) Pub. No.: US 2004/0152631 A1
(43) Pub. Date: Aug. 5, 2004

(54) Methods for manipulating ejaculatory reflex and sensation of ejaculation and for treating sexual dysfunction

(76) Inventor: Lique M. Coolen, Milford, OH (US)

Correspondence Address: DINSMORE & SHOHL, LLP, 1900 CHEMED CENTER, 255 EAST FIFTH STREET, CINCINNATI, OH, 45202, US

(21) Appl. No.: 10/652,788

(22) Filed: Aug. 29, 2003

Related U.S. Application Data
(60) Provisional application No. 60/406,827, filed on Aug. 29, 2002.

(51) Int. Cl. A61K 38/17; A61K 31/198

(52) U.S. Cl. 514/12; 514/566

(57) ABSTRACT

Methods for manipulating sensation of ejaculation in an individual comprise administering to an individual a drug which interact with lumbar spinothalamic (LSt) cells and/or target cells of LSt cells to manipulate sensation of ejaculation in an individual. Methods for manipulating ejaculatory reflex in an individual comprise administering to an individual a drug which interact with lumbar spinothalamic (LSt) cells to manipulate ejaculatory reflex in an individual. Methods for treating sexual dysfunction in an individual comprise administering to an individual a drug which interact with lumbar spinothalamic (LSt) cells to treat sexual dysfunction in an individual.

### RELATED APPLICATION

This application claims priority under 35 U.S.C. §119 of U.S. Provisional Application Serial No. 60/406,827 filed Aug. 29, 2002.

### GOVERNMENT INTERESTS

This invention was made, at least in part, with funds from the Federal Government, awarded through grant number R01 MH60781. The U.S. Government therefore has certain acknowledged rights to the invention.

### FIELD OF THE INVENTION

The present invention is directed toward methods for manipulating sensation of ejaculation in an individual. The invention is also directed toward methods for manipulating ejaculatory reflex in an individual. Additionally, the invention is directed toward methods for treating sexual dysfunction in an individual.

### BACKGROUND OF THE INVENTION

Male sexual behavior is a complex behavior dependent on several intrinsic and extrinsic factors, including olfactory, somatosensory, and visceral cues. Owing to various conditions and variations in ejaculation, it would often be advantageous to manipulate sensation of ejaculation in an individual. Moreover, it would be advantageous to manipulate ejaculatory reflex in an individual. Furthermore, it would be advantageous to treat sexual dysfunction in an individual.

### SUMMARY OF THE INVENTION

Accordingly, it is an object of the invention to provide methods for manipulating sensation of ejaculation in an individual. It is a further object of the invention to provide methods for manipulating ejaculatory reflex in an individual. It is yet a further object of the invention to provide methods for treating sexual dysfunction in an individual.

In accordance with one aspect of the invention, there are provided methods for manipulating sensation of ejaculation in an individual. The methods comprise the step of administering to an individual a drug which interacts with lumbar spinothalamic (LSt) cells and/or target cells of LSt cells to manipulate sensation of ejaculation in the individual.

In accordance with another aspect of the invention, there are provided methods for manipulating ejaculatory reflex in an individual. The methods comprise the step of administering to an individual a drug which interacts with lumbar spinothalamic (LSt) cells to manipulate ejaculatory reflex in the individual.

In accordance with yet another aspect of the invention, there are provided methods for treating sexual dysfunction in an individual. The methods comprise the step of administering to the individual a drug which interacts with lumbar spinothalamic (LSt) cells to treat the sexual dysfunction.

The present invention is advantageous for providing methods for manipulating sensation of ejaculation in an individual. The present invention is also advantageous for providing methods for manipulating ejaculatory reflex in an individual. The present invention is also advantageous for providing methods for treating a sexual dysfunction in an individual. Additional embodiments, objects and advantages of the invention will become more fully apparent in view of the following detail description.

### DETAILED DESCRIPTION OF THE DRAWINGS

The following detailed description will be more fully understood in view of the drawings comprising:

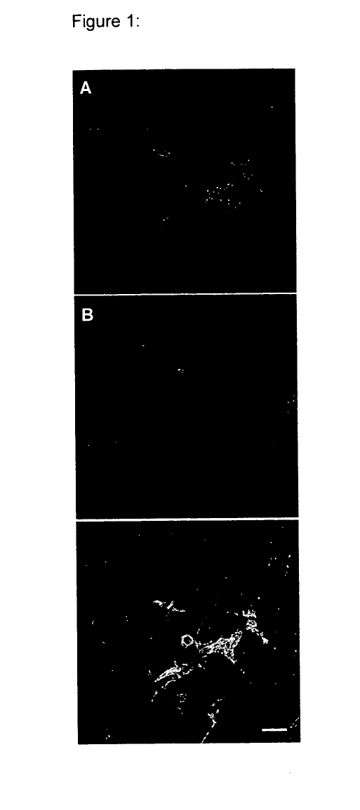

FIG. 1 is an illustration of the co-expression of galanin (A) and SPR (B) in LSt neurons with fluorescent images. Scale bar 50 μm;

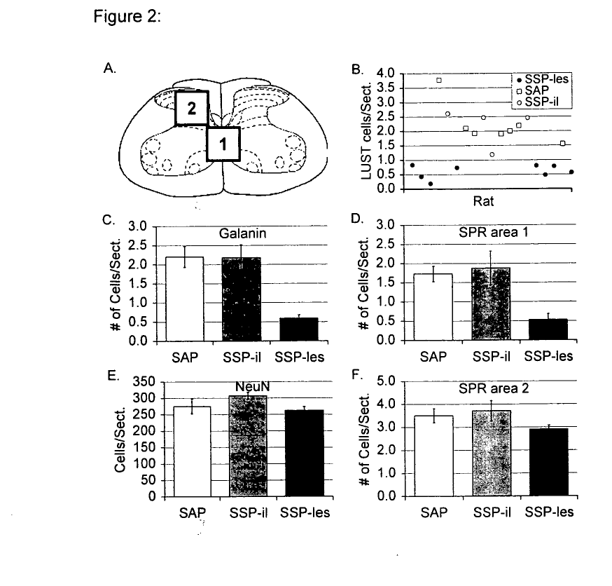

FIG. 2 is an illustration of quantitative analysis of galanin-IR (B, C), Substance P receptor-IR (D, F), and neuron marker N-IR (NeuN, E) cells. Counts are performed in two separate areas in L3 and L4 (A; 800×800 μm each). Counts illustrated in B-E are performed in area 1, which includes the location of LSt cells. B illustrates the average number of LSt neurons per analyzed tissue section for each animal (average of 15.21±0.99 sections are analyzed per animal), demonstrating the effectiveness of lesions within the three groups (SSP-les, n=8; SSP-il, n=4; SAP, n=7). SSP-les animals have less than 0.906±0.058 LSt neurons per section, versus 2.709±0.174 neurons per section in untreated animals. In graphs C—F group means±SEM are shown of the average number of cells per analyzed section per animal. SSP-les rats have significantly less LSt neurons evidenced by a reduction in numbers of galanin-IR (C) or SPR-IR (D) neurons compared to SSP-il or SAP rats. In contrast, there is no significant reduction in NeuN-IR (E), or SPR-IR in the dorsal horn (F; counts performed in area 2 (A)). * p≦0.001 compared to SAP and SSP-il.

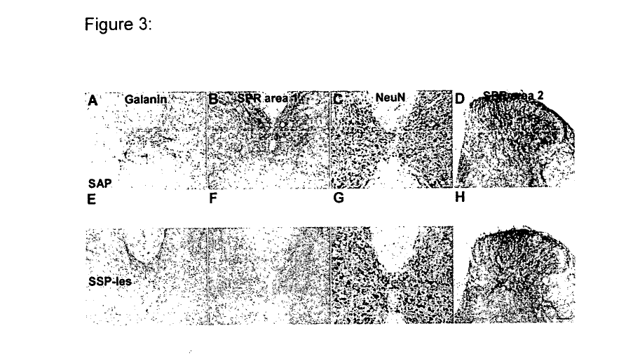

FIG. 3 is an illustration of photomicrographs of Galanin-IR (A, E), Substance P receptor-IR (SPR-I; B, F), and neuronal marker N-IR (NeuN; C, G) in area 1 surrounding the central canal. In addition, Substance P receptor-IR is shown in area 2 (SPR-2; D, H) in the dorsal horn. All photomicrographs illustrate L4 of a representative SAP (A, B, C, D) and SSP-les (E, F, G, H) animal. Galanin and SPR-IR are visibly reduced, while NeuN labeling shows no reduction in SSP- vs. SAP-treated males. Scale bar 100 μm.

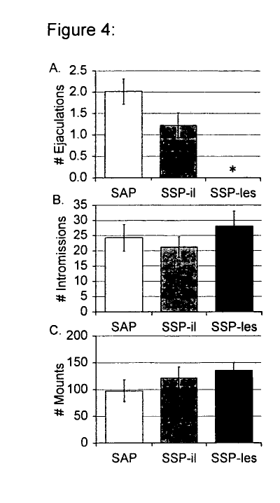

FIG. 4 is a graphical illustration of the quantitative analysis of sexual behavior in which LSt neuronal lesioning abolishes ejaculatory behavior but has no effect on number of mounts or intromissions. Group means±SEM of numbers of ejaculations (A), intromissions (B), and mounts (C). Numbers are averaged across 6 post-surgery trials. * p≦0.001 compared to SSP-il or SAP. No differences are detected between SAP and SSP-il animals (p>0.05).

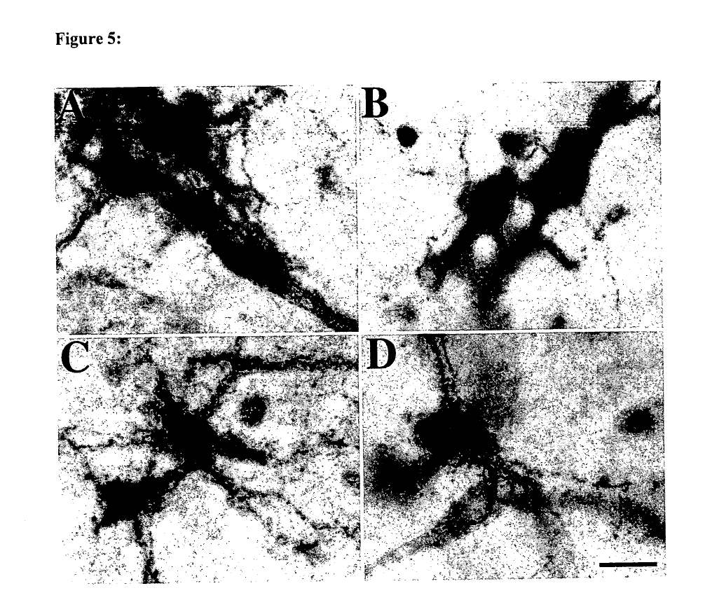

FIG. 5 is a graphical illustration of neural activation of LSt neurons in male, but not female spinal cord. (A) galanin neurons are Fos positive in representative male after 2 ejaculations; (B) lack of co localization of Fos and galanin in male rat following intromissions but no ejaculation; (C) co localization or galanin and Fos in 8-OH-DPAT-treated male rat following 1 ejaculation; (D) lack of co localization of Fos and galanin in female rat following ejaculation by male partner. Scale bar indicates 20 μm.

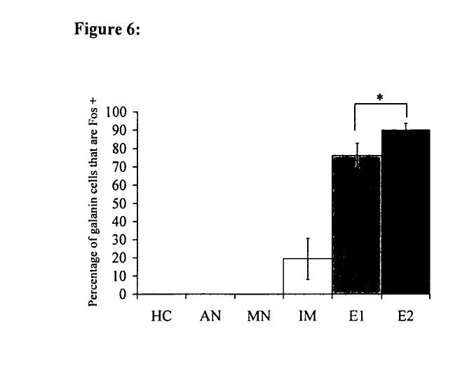

FIG. 6 is a graphical illustration of the percentage of galanin-IR neurons that are Fos-IR. Mean percentages±SEM of galanin-IR cells that are Fos-IR per behavioral group (AN, males that are placed with an anestrous female; MN, males that only mount; IM, males that mount and display intromissions; E1, males that display copulatory behavior including one ejaculation; E2, males that display copulatory behavior including two ejaculations). * different from all other groups, p<0.0001.

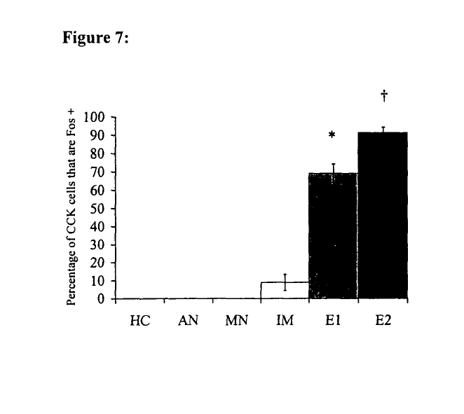

FIG. 7 is a graphical illustration of the percentage of CCK-IR neurons that are Fos-IR. Mean percentages±SEM of CCK-IR cells that are Fos-IR per behavioral group. * different from all other groups, p<0.0001, † greater than E1, p=015.

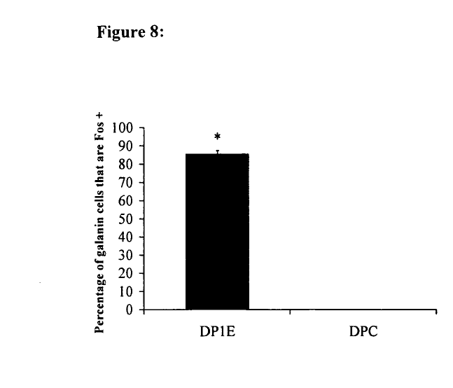

FIG. 8 is a graphical illustration of the percentage of galanin-IR neurons that are Fos-IR following 8-OH-DPAT treatment. Mean percentages±SEM of galanin-IR neurons expressing Fos in rats injected with 0.8 mg/kg of 8-OH-DPAT either after copulation to one ejaculation (DP1E; n=3) or without exposure to female (DPc; n=3), p≦0.0001.

### DETAILED DESCRIPTION OF THE INVENTION

Neural regulation of sexual behavior in male rodents is based largely on studies of copulatory behavior following manipulations of specific brain areas. In recent years, the neural circuits underlying male sexual behavior have been investigated using c-Fos expression as a marker for neural activation. A number of these studies have demonstrated the existence of a subcircuit within the larger circuits underlying male sexual behavior, in which neural activation is solely expressed following ejaculation, but not following intromissions. Activation of this subcircuit is specifically related to ejaculation and not other aspects of sexual activity. The subcircuit in which ejaculation-related Fos is expressed consists of small regions within subdivisions of medial amygdala (MEA), bed nucleus of the stria terminalis (BNST), and the medial portion of the parvocellular subparafascicular thalamic nucleus (mSPFp).

Ejaculation-induced Fos in the posterior thalamus is restricted to a medial portion of the parvocellular subparafascicular nucleus (SPFp). The thalamus is known to receive direct sensory inputs from the spinal cord, and may thus be an important relay for genital sensory inputs to other areas of the brain important for sexual behavior. Suprisingly, in female rats, Fos-IR following vaginocervical stimulation has a similar distribution as the ejaculation-induced Fos-IR in male rats. While not wishing to be bound by theory, the inventor believes that neural activation in this subcircuit is associated with processing of genital sensory signals specifically related to ejaculation or vaginocervical stimulation.

The inventor has discovered that the mSPFp, the posterior thalamic area expressing ejaculation-related Fos, receives a unique input from a specific subpopulation of spinothalamic neurons in the lumbar spinal cord. The inventor has further discovered that this population of neurons is located in laminae 7 and 10 of the Lumbar (L) segments 3 and 4. This population of lumbar SPFp-projecting neurons is referred to as LSt (Lumbar spinothalamic) cells. LSt cells are specifically activated following ejaculation, but not following other components of male sexual behavior.

Accordingly, the inventor has discovered that lumbar spinothalamic (LSt) cells) may be used to manipulate sensation of ejaculation and ejaculator reflex in an individual, as well as to treat a sexual dysfunction in an individual. In one embodiment, the present invention is directed towards methods for manipulating sensation of ejaculation in an individual. The methods comprise the step of administering to an individual a drug which interacts with lumbar spinothalamic (LSt) cells and/or target cells of LSt cells to manipulate sensation of ejaculation in the individual. In a further embodiment, the invention is directed towards methods for manipulating ejaculatory reflex in an individual. The methods comprise the step of administering to an individual a drug which interacts with lumbar spinothalamic (LSt) cells to manipulate ejaculatory reflex in the individual. In yet a further embodiment, the invention is directed toward methods for treating a sexual dysfunction. The methods comprise the step of administering to an individual a drug which interacts with lumbar spinothalamic (LSt) cells to treat a sexual dysfunction in the individual.

Within the scope of the present specification and claims, the term “interacts” means that the activity of the LSt cells is altered by the administration of the drug. In specific embodiments discussed in further detail below, the drug may act as an agonist on the LSt cell, as an antagonist on the LSt cells, or may exhibit other altering effects.

Spinothalamic neurons have been associated with signaling pain and temperature information. The inventor has determined that a specific subpopulation of spinothalamic neurons, comprised of galanin- and CCK-positive lumbar spinothalamic neurons, are involved in the signaling of sensory information pertaining to ejaculation. These LSt neurons project to the mSPFp, a brain region that has been previously reported to express Fos following ejaculation or vaginocervical stimulation in male and female rodents. However, the present findings showed that the LSt neurons are only activated in male rats with ejaculation and not with vaginocervical stimulation in female rats. While not wishing to be bound by theory, the inventor believes that these results suggest that this spinothalamic pathway is involved in the relay of genital sensory information selectively related to ejaculation.

Activation of the LSt neurons is triggered by sensory stimuli associated with ejaculation and not other aspects of sexual behavior, since Fos expression is observed following ejaculation but not mounts or intromissions. Intromissions require penile erection, which, in turn, include relaxation of smooth muscles and activation of striated muscles involved in flips, engorgement of the glans penis and straightening of the penis. Intromission additionally includes superficial stimulation of the penis upon insertion into the vagina. Animals that ejaculate have two additional phases, emission and ejaculation. Both of these phases may be associated with unique sensory stimuli. Emission consists of moving seminal fluids into the proximal urethra and involves sympathetically mediated contraction of vas deferens and epididymis, and parasympathetically mediated contractions of the prostate. The expulsion phase of ejaculation is the forceful emptying of the urethral contents and is accompanied by rhythmic bursting of the ischiocavernosus and bulbocavemosus muscles, as well as, urethral dilation, contractions of the bladder neck, internal urinary sphincter and anal sphincter. Therefore, afferent genital sensory information accompanying ejaculation may be composed of somatosensory, visceral sensory, and proprioceptive signals.

The location of the LSt neurons is consistent with relay of genital sensory information to higher levels of the CNS. Genital sensory information is conveyed by one or more of three different nerves, the pudendal, hypogastric, and pelvic nerve. The majority of the sensory fibers in these nerves enter the spinal cord at levels T12-L2 (hypogastric) and L6-S3 (pelvic and pudendal). However, some primary afferents project in Lissauer's tract rostral and caudal to the entry zone. These afferents extend rostrally as far as the rostral part of L3, where they tend to concentrate in the medial portion of Lissauer's tract. This rostrally-directed pathway may be involved in transmission of sensory information from the periphery.

The LSt neurons are therefore ideally situated to receive sensory inputs and possibly integrate information arising from any or all of these nerves. Furthermore, LSt neurons are located in lamina 7 and 10, an area reported to receive afferents from viscera and muscles, which are likely candidates for sensory inputs unique to ejaculation.

Activation of the LSt neurons may induced by sensory stimuli associated with events before, during, or after ejaculation. In support of activation occurring immediately before ejaculation, Fos is induced in LSt neurons in three of the ten rats displaying intromissions but not ejaculation. Interestingly, these males displayed atypical mounting behavior that resembles the onset of ejaculatory behavior and is often immediately followed by display of ejaculation (personal observations). Thus, it is possible that these males ejaculated in the absence of a female. Alternatively, it is possible that a summation of sensory events prior to ejaculation results in a state of readiness for ejaculation, in humans often referred to as “ejaculation inevitability”. It has been suggested that “ejaculation inevitability” is associated with emission, and that the stimuli unique to emission may subsequently trigger ejaculation. Thus, LSt neurons may receive sensory input associated with emission. In addition, activation of the LSt neurons may be a result of visceral sensory inputs associated with expulsion of the seminal fluids. Finally, activation the LSt neurons may be associated with events that occur after ejaculation such as auto grooming or sexual inactivity.

Although activation of LSt neurons may result from a summation of sensory stimuli unique to emission or ejaculation, summation of sexual activity normally preceding ejaculation is not required to elicit Fos expression.

LSt neurons are ideally situated to relay genital sensory information to the mSPFp. Ejaculation results in neural activation of LSt neurons as well as in mSPFp. However, while not wishing to be bound by theory, the inventor believes that the activation of LSt neurons is causally related to activation of neurons in the mSPFp. In addition, while not wishing to be bound by theory, the inventor believes that the neuropeptides localized in LSt neurons are involved in activation of mSPFp neurons.

Vaginocervical stimulation in female rats induces Fos expression in the mSPFp but not in LSt neurons. While not wishing to be bound by theory, the inventor believes that it is possible that sensory information related to female sexual function is relayed to the mSPFp and other areas of the brain via a pathway different than that of the male and does not include the LSt neurons.

Obviously, based on a lack of Fos expression it cannot be concluded that these LSt neurons are not involved with sexual behavior in the female, and other markers of activation may indeed demonstrate that these neurons are activated following female sexual behavior.

While not wishing to be bound by theory, the inventor believes that the activation of LSt neurons in males is a consequence of ejaculation. Subsequently, activation of these neurons could trigger ejaculation. Indeed, the inventor demonstrated that selective lesions of LSt cells completely disrupted ejaculatory behavior while not effecting other components of male sexual behavior. The pathways via which LSt neurons may be crucially involved in ejaculation may include local projections to preganglionic or motor neurons controlling muscles associated with emission or ejaculation, or supraspinal projections to brain regions that in turn can control ejaculation.

Accordingly, the inventor has discovered that lumbar spinothalamic (LSt) cells) and/or their target cells may be used to manipulate sensation of ejaculation. In one embodiment, the present invention is directed towards methods for manipulating sensation of ejaculation in an individual. The methods comprise the step of administering to an individual a drug which interacts with lumbar spinothalamic (LSt) cells and/or target cells of LSt cells to manipulate sensation of ejaculation in the individual.

As used herein, “manipulating” is intended to refer to the enhancement or diminution in an individual of sensation of ejaculation, ejaculatory reflex or combinations thereof. As used herein, “individual” is intended to refer to an animal, including but not limited to mammals, including humans and rodents.

In one embodiment, the LST cells are positioned in lamina XII and X in lumbar segments 3 (L3) and 4 (L4). In a further embodiment, the LST cells for manipulating the sensation of ejaculation comprise neurotransmitters, neuropeptides, or combinations thereof. The neurotransmitters include, but are not limited to, galanin, cholecystokinin, enkephalin, glutamate, or combinations thereof.

One skilled in the art will appreciate the various known techniques for administering a drug to an individual, any of which may be used herein. The known techniques include, but are not limited to, oral, parenterally, intrasystemically, intraperitoneally, topically, or combinations thereof. Moreover, one skilled in the art will appreciate the various known drugs which interact with lumbar spinothalamic (LSt) cells, any of which may be used herein. In one embodiment, the drug comprises an agonist of at least one neurotransmitter. In another embodiment, the drug comprises an antagonist of at least one neurotransmitter.

In addition, to genital sensory signals, ejaculation is considered to be a reflex and the central components necessary to complete this reflex are located in the lumbosacral spinal cord. In particular, the ejaculatory reflex remains intact in spinalized individuals and may be evoked by electrostimulation or intense vibration in humans with spinal cord injuries between cervical level 3 and lumbar level 3.

The inventor has discovered evidence that the population of lumbar spinothalamic (LSt) neurons, which manipulate genital sensory information also play a pivotal role in generation of ejaculatory behavior, suggesting that these LSt cells form at least a part of the spinal ejaculation generator. The spinal ejaculation generator is a system that coordinates somatic, sympathetic, and parasympathetic outflow to induce emission and expulsion. This response is produced in response to stimulation during sexual activity and sexual arousal. The spinal ejaculation generator is under inhibitory and excitatory descending control from supraspinal sites in brain and brainstem.

Moreover, LSt cells have additional characteristics that appear essential to an ejaculation generator. Ejaculation consists of two phases, emission (secretion and movement of seminal fluids to the urethra) and expulsion (forceful ejection of urethral contents), which have been demonstrated to follow a highly synchronized series of events.

The ejaculatory reflex is complex and modular in nature and involves multiple efferent and afferent systems. Stimulation to induce ejaculation may involve visceral, proprioceptive and somatic inputs. Indeed, LSt cells are activated specifically with ejaculation, but not with intromissions alone. Thus, LSt neurons appear to receive sensory inputs related to ejaculation. In addition, the ejaculatory reflex involves a complex integration of sympathetic, parasympathetic and somatic efferent systems. While not wishing to be bound by theory, the inventor believes that these systems are coordinated by a central pattern generator, consisting of interneurons located in the lumbosacral spinal cord, which produces a sequential rhythmic bursting of muscles and nerves responsible for the secretion and the external ejection of seminal fluid.

Consistent with this hypothesis, data displayed in Table 1 indicate that LSt neurons have direct projections to sympathetic neurons of the intermediolateral column and the central autonomic nucleus, areas that are crucial to the emission phase of ejaculation, and to the sacral parasympathetic nucleus involved in regulation of epithelial secretion and prostatic activity, as well as sphincter control.

The data displayed in Table 1 are optical density measurements in pixels (×1,000) of galanin-IR or Substance P receptor-IR (SPR) in areas surrounding the intermediolateral column (IML), central autonomic nucleus (CAN), and sacral parasympathetic nucleus (SPN). In these areas galanin-IR is present in fibers, while SPR-IR is located in neurons and fibers. SSP-les animals have significantly reduced galanin-IR, indicating that the galanin-IR fibers in these areas are the axon terminals of LSt neurons. In contrast, SPR-IR is not reduced following LSt lesions. Thus, while not wishing to be bound by theory, the inventor believes that these results indicate that loss of galanin-IR fibers in these areas is not a result of spread of the toxin, but rather reflects the loss of LSt axon terminals. * p≦0.001 compared to SAP and SSP-il.

TABLE 1

| | SAP | SSP-il | SSP-les |
|---|---|---|---|
| IML-galanin | 58.82 ± 12.85 | 70.87 ± 18.58 | 20.47 ± 5.61* |
| CAN-galanin | 128.84 ± 25.81 | 118.13 ± 21.25 | 25.56 ± 9.41* |
| SPN-galanin | 137.75 ± 14.99 | 177.60 ± 26.40 | 77.64 ± 10.79* |
| IML-SPR | 209.91 ± 30.92 | 150.81 ± 71.11 | 176.83 ± 21.44 |
| CAN-SPR | 380.26 ± 39.06 | 266.99 ± 159.47 | 241.75 ± 49.26 |
| SPN-SPR | 392.68 ± 50.65 | 362.91 ± 101.08 | 340.97 ± 49.89 |

Accordingly, the inventor has discovered that lumbar spinothalamic (LSt) cells) may be used to manipulate ejaculatory reflex in an individual. In one embodiment, the invention is directed towards methods for manipulating ejaculatory reflex in an individual. The methods comprise the step of administering to an individual a drug which interacts with lumbar spinothalamic (LSt) cells to manipulate ejaculatory reflex in the individual.

Methods for manipulating ejaculatory reflex in an individual comprise the step of administering to an individual a drug which interacts with lumbar spinothalamic (LSt) cells to manipulate ejaculatory reflex in an individual. In one embodiment, the LSt cells are positioned in lamina XII and X in lumbar segments 3 (L3) and 4 (L4). In a further embodiment, the LSt cells for manipulating ejaculatory reflex comprise neurotransmitters, neuropeptides, or combinations thereof. The neurotransmitters include, but are not limited to, gamma-amino-butyric-acid. The neuropeptides include, but are not limited to, substance p, serotonin, somatostatin, galanin, vasointestinal peptide, calcitonin gene related peptide, or combinations thereof.

One skilled in the art will appreciate the various known techniques for administering a drug to an individual, any of which may be used herein. The known techniques include, but are not limited to, oral, parenterally, intrasystemically, intraperitoneally, topically, or combinations thereof. Moreover, one skilled in the art will appreciate the various known drugs which interact with lumbar spinothalamic (LSt) cells, any of which may be used herein. In one embodiment, the drug comprises an agonist of at least one neurotransmitter. In another embodiment, the drug comprises an antagonist of at least one neurotransmitter.

The present invention is also directed to methods for treating sexual dysfunction in an individual. The methods comprise the step of administering to an individual a drug which interacts with the lumbar spinothalamic (LSt) cells to treat sexual dysfunction. Sexual dysfunction includes, but not limited to premature ejaculation, delayed ejaculation, control of ejaculatory function in an individual with spinal cord injury, anorgasmic ejaculation, or combinations thereof.

The methods will be more fully understood in view of the examples.

### EXAMPLES

### Example 1

To test the behavioral significance of LSt neurons, effects of lesions of the LSt population on sexual behavior are investigated. LSt neurons are sparsely distributed lateral to the central canal in lamina VII and X of L3 and L4, and thus difficult to lesion by traditional methods. Therefore, a targeted toxin approach is utilized.

First a membrane target located on the LSt neurons is identified. As illustrated in FIG. 1, 93.0±1.7% of LSt neurons express substance P receptor (SPR) and conversely 84.7±2.47% of SPR containing cells in the area surrounding the central canal at L3-4 express galanin,. Identification of the SPR receptor on LSt cells enable the use of the targeted toxin SSP-SAP, which consists of the ribosome inhibitor protein toxin saporin (SAP) conjugated to a substance P analogue (SSP) with high affinity for SPR. SSP-SAP is infused into the L3-4 spinal cord at the location of the LSt cells in sexually experienced or sexually naive male rats. Male rats are allowed to mate with a receptive female during 6 pre-operative sessions (3 ejaculations or 100 minutes each).

All procedures are approved by the Animal Care and use Committee of the University of Cincinnati, Ohio and conformed to NIH guidelines involving vertebrate animals in research. Male rats are deeply anesthetized using ketamine/xylazine and laminectomy of L3 and L4 is performed. SSP-SAP (4 ng/μl) is microinjected into lamina X of L3-4 in a series of 6-8 bilateral 1 μl injections. Following surgery, the wound is covered with gelfoam and closed.

Control animals are injected with non-conjugated equimolar concentrations of SAP. The doses used have been previously demonstrated to selectively ablate SPR containing cells in vivo without producing nonselective lesions. Rats with obvious motor impairment after surgery (n=10; 7 injected with SAP; 3 injected with SSP-SAP) are excluded from the study. Sexual behavior is first tested 10 days after lesion surgery, and during 5 subsequent twice-weekly tests. The trials are performed during the dark phase of the light cycle and consisted of the male rat being placed into the testing cage for 100 minutes with a receptive female. Stimulus females were ovariectomized and implanted subcutaneously (s.c.) with 5% 17-beta-estradiol benzoate silastic capsules. Progesterone (500 μg in 0.1 ml of sesame oil) was injected (s.c.) 4-6 hours prior to testing to induce sexual receptivity. Latencies and numbers of mounts, intromissions, and ejaculations are recorded, and vaginas of females are checked for seminal plugs multiple times during mating tests.

Following the final behavior test, animals are perfused intracardially with 4% paraformaldehyde and spinal cord tissue is collected for further processing. Spinal cords are cut into 35μm coronal sections and collected in 12 parallel series. Adjacent sections are stained for galanin, SPR or neuronal marker N using the general procedures as described in W. Truitt, M. Shipley, J. Veening, L. Coolen, J. Neurosci (2002). The primary antibodies and concentrations that are used include: Galanin—rabbit anti-galanin at 1:80,000 (PenLabs), Substance P receptor—rabbit anti-NK1 at 1:1,000 (Advanced Targeting Systems), and Neuronal marker N—mouse anti-NeuN at 1:10,000 (Chemicon).

Labeled cells in a standard area surrounding the central canal of L3 and L4 sections, as illustrated in FIG. 2A (area 1), are counted to determine if treatment reduced the number of LSt neurons visualized by galanin immunoreactivity (-IR) or SPR-IR. Non-specific lesioning is determined by counting neurons visualized by neuronal marker N-IR. SPR containing cells are also counted in a standard area in the dorsal horn, as illustrated in FIG. 2A (area 2), to determine if lesioning is restricted to the area surrounding the central canal.

A lesion is considered complete if the number of galanin-IR LSt neurons is less than ⅓ of the number of LSt cells observed in untreated rats. Untreated male rats (n=6) had 2.709±0.174 (mean±SEM) of galanin-IR cells per L3-L4 section. Rats with less than 0.906 galanin-IR cells per L3/L4 section are considered completely lesioned. Of the 19 rats included in the behavioral analysis, 8 SSP-SAP-treated rats had complete lesions of LSt neurons (SSP-les), as illustrated in FIGS. 2B and 3, while incomplete or misplaced lesions are observed in 4 SSP-SAP treated males (SSP-il), and no lesions are present in SAP treated males (SAP, n=7). SSP-les animals have significantly fewer galanin-IR neurons than SSP-il or SAP animals (p<0.001; all statistical analyses data are compared using a one-way ANOVA and posthoc comparisons are made using the Scheffe test, both with a 0.05 level of significance), as illustrated in FIGS. 2C and 3.

Cell counts performed in adjacent sections immunostained for SPR further confirms these data. SSP-les rats have a significantly reduced number of SPR containing cells (p<0.006), as illustrated in FIGS. 2D and 3, compared to SSP-il, or SAP animals. Despite the severe reduction in LSt neurons in SSP-les rats there is no overall reduction in numbers of NeuN-IR cells (p>0.26), illustrated in FIGS. 2E and 3, indicating the selectivity of the lesions. The lesions are restricted to the area surrounding the central canal and did not affect the number of SPR expressing neurons in the dorsal horn, as illustrated in FIG. 2F.

In addition, pain perception is not altered in SSP-les animals as compared to SSP-il or SAP controls. Pain thresholds are assessed in rats from all treatment groups (SSP-les n=4, SSP-il n=4 and SAP n=6). Rats are placed on a hot plate (52° C.) until licking of a hind paw is recorded. Rats did not remain on hot plate longer than 60 seconds. The process is repeated 5 times with 10 minutes between each trial. Data is averaged across the five trials for each animal, and group means are calculated. No significant difference is found between groups, (mean±S.E.M. were 33.33±2.52 for SAP rats, 35.98±3.78 for SSP-il, and 33.32±2.52 for SSP-les; repeated measures ANOVA, p>0.85).

The effects of LSt lesions on display of sexual behavior are assessed in the three groups described above. LSt lesions have a dramatic effect on sexual behavior. Lesions completely disrupted display of ejaculatory behavior in SSP-les males and seminal plugs are uniformly absent upon examination of the female partner throughout the testing session. In contrast, SSP-il and SAP males continue to ejaculate regularly following surgery, as illustrated in FIG. 4A. Furthermore, ablation of LSt neurons appears to selectively block ejaculatory behavior while not affecting other components of sexual behavior. SSP-les animals did not differ from the SSP-il or SAP animals in number of mounts (p>0.317) or intromissions (p>0.644), as illustrated in FIGS. 4B and 4C. Average mount latencies, and average intromission latencies also did not significantly different among groups (p>0.492; p>0.569 respectively). These results indicate that LSt neurons are essentially involved in the onset of ejaculatory behavior.

There are no detectable differences in behavior between animals that are sexually experienced or naive prior to lesion surgery, thus experience did not play a role in the effects of LSt cell lesions. Hence, data is shown grouped for sexually naive and experienced animals.

### Example 2

Activation of LSt neurons is assessed using Fos immunoreactivity (IR) as a marker for neural activation and galanin or CCK-IR to identify LSt cells. The specificity of Fos expression in LSt cells for ejaculation is assessed by comparing Fos induction by sexual behaviors that did not culminate in ejaculation.

Young adult male (n=55; 250-260 grams) and female Sprague Dawley rats (n=19; 210-220 grams) obtained from Harlan labs (Indianapolis, Ind.) are housed in same-sex pairs in artificially lighted rooms on a reversed 12:12 light dark cycle (lights off at 10 AM). Food and water are available at all times. Stimulus females are ovariectomized and implanted subcutaneous (s.c.) with 5% 17-beta-estradiol benzoate silastic capsules. Progesterone, 500 μg in 0.1 ml of sesame oil is injected (s.c.) 4-6 hours prior to testing to induce sexual receptivity. All procedures are approved by the Animal Care and use Committee of the University of Cincinnati and conformed to NIH guidelines involving vertebrate animals in research.

All male and female rats are sexually experienced. Male rats are allowed to copulate during six pre-test mating sessions (30 minutes duration) and are considered sexually experienced when they displayed multiple ejaculations during the last two mating sessions. The female rats are bilaterally ovariectomized two weeks prior to testing. Sexual receptivity is induced using a standard regimen of exogenous estrogen and progesterone, similar to that used for the stimulus females, described above. One week before testing females receive hormone treatment and mating experience with sexually experienced male partners. All testing of experimental groups is performed four hours after onset of the dark period, in a rectangular mating arena (60-45-50 cm), under dim red illumination. After completion of the mating tests, the sexual partners are removed, and experimental animals remained in the test cage for an additional 60 minutes until sacrifice.

Perfusion, Tissue Processing and Immunocytochemistry

One hour following the end of the behavioral test, animals are anesthetized using sodium pentobarbital (200 mg/kg; i.p.) and perfused transcardially with 100 ml 0.9% saline, followed by 500 ml of 4% paraformaldehyde in 0.1 M sodium phosphate buffer (PB, pH 7.3-7.4). Brain and spinal cords are removed and post fixed for 1 hr at room temperature in the fixative. Coronal sections of thoracic (T), lumbar (L) and sacral (S) spinal cord (T10-S4) are cut at 35 μm on a Microm freezing microtome (Richard Allen, Kalamazoo, Mich.) and collected in twelve parallel series in cryorotectant solution (30% sucrose, 30% ethylene glycol in 0.1 M PB; Watson et al, 1985). In addition, coronal sections ( 35 □m) through thalamus are cut and stored in four parallel series.

All incubations are performed at room temperature with gentle agitation. Series of spinal cord sections (420 m apart) are rinsed extensively in phosphate buffered saline (PBS) between incubations. Free-floating sections are blocked with 1% H₂O₂ for 10 min at room temperature and then soaked for one hour in incubation solution (PBS containing 0.1% bovine serum albumin and 0.4% triton X-100). Next, sections are incubated overnight with a primary antiserum in incubation solution to recognize Fos (polyclonal anti-Fos antiserum raised in rabbit; 1:10,000; Santa Cruz, Santa Cruz, Calif.). Subsequently, sections are exposed for 60 minutes to biotin-conjugated donkey anti-rabbit IgG, (1:400 in incubation solution, Jackson Immunoresearch, Westgrove, Pa.) and for 60 minutes to avidin-biotin-horseradish peroxidase (ABC-elite, 1:1,000 in PBS; Vector Laboratories, Burlingame, Calif.). The peroxidase complex is visualized by exposure for 10 minutes to a chromogen solution containing 0.02% 3,3′-diaminobenzidine tetrahydrochloride (DAB, Sigma, St. Louis, Mo.) enhanced with 0.02% Nickel sulfate in 0.1 M PB with hydrogen peroxide (0.015%) to produce a blue-black reaction product. Extensive washing in 0.1M PBS terminates the reaction.

The sections are then blocked with 1% H₂O₂ for ten minutes at room temperature, and incubated overnight with primary antiserum in incubation solution, to recognize galanin (polyclonal anti-galanin antiserum raised in rabbit: 1:80,000; PenLabs, San Carlos, Calif.), or CCK (polyclonal anti-CCK raised in rabbit: 1:30,000; Chemcom Temecula, Calif.). Next, sections are processed using the same avidin-biotin immunoperoxidase procedure described above with the second chromogen reaction performed without Nickel sulfate, to produce a brown reaction product. Sections are mounted on superfrost plus glass slides (Fisher), dried overnight and then dehydrated with a series of alcohol and Hemo-D (Fisher) washes and cover-slipped with DPX (Electron Microscopy Sciences, Fort Washington, Pa.). Immunocytochemical controls include omission of primary antibodies for Fos, galanin, or CCK.

Data Analysis Sexual Behavior

The number of mounts, intromissions and total duration of the tests, are recorded for analysis of copulatory behavior. Numbers of mounts and intromissions, as well as duration of test are compared using a one-way ANOVA. Post hoc comparisons are made using the Scheffe test with an alpha of 0.05 required for rejection of the null hypothesis.

Immunocytochemistry

Analysis of labeling for galanin or CCK, Fos, and dual labeling for Fos and Galanin or CCK, is performed on a Leica light microscope at 4×-40× magnification. Neurons single labeled for galanin or CCK are determined by brown staining of cytoplasm with a nucleus devoid of blue-black (Fos) reaction product. Neurons with only blue-black nuclei are considered single-labeled for Fos. Cells that contain both stained nucleus (blue-black) and cytoplasm (brown) are considered dual labeled. Dual labeling is expressed as the percentage of galanin or CCK cells that expressed Fos. Group means are based on percentage of double-labeled cells for individual rats. To determine differences between groups a one-way ANOVA and post hoc comparisons using the Scheffe test are performed, both with a 0.05 level of significance. A Spearman's rank correlation test was performed to determine if a correlation exists between behavior (duration, number of mounts or number of intromissions) and the percentage of double-labeled neurons, using a 0.05 level of significance.

### Example 2A

This example, using the methods described in Example 2 in detail above, demonstrates the activation of spinothalamic neurons in male rats. Activation of LSt neurons is investigated in male rats following different elements of sex behavior. Male rats are divided in five experimental groups and one control group, in order to study induction of Fos-IR after different components of sexual behavior. The male control group consists of males sacrificed immediately after removal from their home cage. The first experimental group consists of males that interacted with an anestrous female (AN; n=9). The second group consists of males that only displayed mounts (MN; n=9). To prevent occurrence of intromissions, the anogenital regions of the stimulus females were covered with masks. The third group consists of males displaying mounts and intromissions, but no ejaculations (IM; n=10). In the fourth group are tested until one ejaculation occurred (E1; n=l 1). And finally in the fifth group, males are tested until two ejaculations had occurred (E2; n=7).

Table 2 summarizes the behavioral data (numbers of mounts and intromissions and total test duration) for all male experimental groups. Overall, there are few differences in the behavioral parameters between groups. Specifically, the numbers of mounts and intromissions did not significantly differ between groups. However, not surprisingly, test duration is longer for E2 males than for E1 or IM males (p=0.0396).

TABLE 2
Summary of male sex behavior by group

| | Mean | Mean | Mean |
|---|---|---|---|
| Males | Mounts | Intromissions | Duration (s) |
| HC | — | — | — |
| (n = 5) | | | |
| AN | — | — | 677.6 ± 66.2 |
| (n = 9) | | | |
| MN | 25.4 ± 2.4 | — | 726.1 ± 75.5 |
| (n = 9) | | | |
| IM | 17.9 ± 2.5 | 13.1 ± 1.9 | 473.6 ± 57.9 |
| (n = 10) | | | |
| E1 | 29.0 ± 2.7 | 15.8 ± 1.1 | 663.1 ± 76.7 |
| (n = 11) | | | |
| E2 | 28.8 ± 4.2 | 19.4 ± 2.3 | 1029.0 ± 102.6† |
| (n = 7) | | | |

Presented in Table 2 are the mean±SEM for number of mounts and intromissions as well as the mean±SEM time spent with a female. † indicates different from IM and E1 (p<0.05)

In all males, galanin-IR neurons are observed in lamina VII and X of L3 and L4 (FIG. 1) and, the mean number of Galanin-IR neurons is similar for all groups, ranging from 28.14±3.39 to 36.30±3.60 (mean±SEM). The percentage of galanin neurons that express Fos is significantly higher in males that ejaculated compared to males that displayed other elements of sexual behavior (FIG. 2; p<0.007). A majority of galanin-IR neurons contain Fos-IR following one ejaculation, as illustrated in FIG. 5; and two ejaculations did not result in any further increase in the percentage of Fos-positive galanin cells, as depicted in FIG. 6. Males engaged in pursuit of an anestrous female (AN) or mounting behavior alone (MN) fail to display any activation of galanin-IR neurons.

In addition, intromissions alone do not result in a significant increase of Fos expression in galanin neurons, as depicted in FIGS. 5 and 6. However, 3 of 10 males in the intromissions alone group (IM) did display some co-localization of galanin and Fos. The percentages of double labeling in these animals are variable (27.5%, 90.2%, and 81.8%) and are not correlated with the number of mounts or intromissions in these individuals (Spearman's rank correlation; mounts p=0.134; intromissions p=0.372). Curiously, these IM males displayed atypical mounting behavior. In contrast, to normal mounts during which males display pelvic thrusting but no insertion, mounts in these three males, resembled the onset of ejaculatory behavior, attaining a horizontal position atop the female but not displaying the deep thrust.

Since LSt neurons co-express galanin and CCK, mating induced activation of these neurons is further assessed using CCK-IR as a marker. The mean±SEM number of CCK-IR neurons does not significantly differ between groups and ranged from 27.71±3.86 to 42.75±6.81. Furthermore, the average number of CCK-IR neurons does not differ from the average number of galanin-IR neurons (35.02±1.78 and 34.20±1.51 respectively). As is the case for the galanin-IR cells, there is a significant increase in the percentage of CCK cells expressing Fos following ejaculation, but not after other components of male sexual behavior (p≦0.0001, FIG. 7). However, in contrast to the galanin-IR neurons, the percentage of the CCK-IR neurons that are Fos-IR is significantly greater in rats with 2 ejaculations than rats with 1 ejaculation (p=0.015). Males engaged in pursuit of an anestrous female (AN) or mounting behavior alone (MN) fail to display any activation of CCK-IR neurons and, the same 3 males of the IM group that show Fos expression in galanin cells also have Fos-positive CCK cells.

### Example 2B

This example, using the methods described in Example 2 in detail above, demonstrates activation of LOST neurons the following 8-OH-DPAT-induced ejaculation. The purpose of this example is to investigate neural activation of lumbar spinal cord in male rats that display minimal sexual activity prior to ejaculation. Therefore, male rats are treated with 8-hydroxy-2-(di-n-propylamino) tetralin (8-OH-DPAT), which decreases the number of mounts and intromissions prior to ejaculation. Experienced male rats receive a subcutaneous injection of 8-OH-DPAT (0.8 mg/kg; Sigma) 30 minutes prior to being placed in the testing arena. Males either remain in the testing arena without exposure to a female (n=3) or are paired with receptive females for one copulatory series including one ejaculation (n=3). Only male rats that display ejaculation within three intromissions are included in this study.

All E1-DP rats ejaculate in three or fewer intromissions, with one rat ejaculating on the first mount. Thus, the average numbers of mounts (2.33±1.20) and intromissions (1.33±0.67) are lower compared to 1E males in Example 2A. As in Example 2A, the mean number of galanin-IR neurons do not differ between groups (33.67±4.06 and 33.33±5.53), nor do the mean number of galanin-IR neurons counted in this experiment differ from Example 2A. Co-localization of Fos-IR and Galanin-IR neurons is observed in all E1-DP males (FIG. 4), with 85.55%±1.76 of Galanin-IR neurons containing Fos-IR. Treatment with 8-OH-DPAT alone (DPc group) do not result in activation of Galanin neurons.

### Example 2C

This example, using the methods described in Example 2 in detail above, demonstrates the absence of Fos expression LSt neurons in female rats. The experimental female groups are divided based on the sexual activity the females received from the male partners. Control females (n=6) receive a male partner that does not display sexual behavior, and consequently these females do not display lordosis behavior. The first female experimental group consisted of females that received mounts, but no intromissions (MN; n=4). To prevent occurrence of intromissions, the genital regions of the male partners are masked. The second experimental group of females receive mounts and intromissions, but no ejaculation (IM; n=5). The final three groups include females that receive one ejaculation (E1; n=4), two ejaculations (E2; n=3), or three-four ejaculations (E3/4; n=3).

Table 3 summarizes the behavioral data for female rats. The experimental groups only differ significantly in the duration of test, with E2 and E3/4 females having significantly greater test duration than all other groups p<0.05). The mean numbers of galanin-IR cells do not differ between groups (ranging from 24.33±1.86 to 37.67±6.17), and the mean number of galanin-IR for females (30.17±1.34) do not differ from that of males (34.12±1.37). Unlike males, female rats show no co-localization of Fos-IR and Galanin-IR in any group. However, Fos-IR is observed in the mSPFp of females receiving intromissions alone or intromissions including ejaculations.

TABLE 3
Summary of female sex behavior by group

| | Mean | Mean | Mean |
|---|---|---|---|
| Females | Mounts | Intromissions | Duration (s) |
| Control | — | — | — |
| (n = 6) | | | |
| MN | 13.8 ± 1.7 | — | 420.0 ± 114.0 |
| (n = 4) | | | |
| IM | 8.2 ± 2.5 | 11.0 ± 1.9 | 696.0 ± 132.0 |
| (n = 5) | | | |
| E1 | 1.8 ± 0.5 | 10.8 ± 1.1 | 840 ± 138.0 |
| (n = 4) | | | |
| E2 | 8.0 ± 4.6 | 17.3 ± 2.0 | 1620 ± 36.0* |
| (n = 3) | | | |
| E3/4 | 9.0 ± 2.9 | 20.0 ± 2.5 | 2238 ± 102.0** |
| (n = 3) | | | |

Presented in Table 3 are the mean ± SEM for number of mounts and intromissions received by the female as well as the mean ± SEM time spent with a male.
*indicates significant difference compared to all other groups except 3-4 ejaculations;
**indicates significant difference compared to all other groups (p < 0.05).

The specific embodiments in the examples described herein are illustrative in nature only and are not intended to be limiting of the claimed compositions and methods. Additional embodiments and variations within the scope of the claimed invention will be apparent to those of ordinary skill in the art in view of the present disclosure.

What is claimed:

1. A method for manipulating sensation of ejaculation in an individual, comprising the step of administering to an individual a drug which interacts with lumbar spinothalamic (LSt) cells and/or target cells of LSt cells to manipulate sensation of ejaculation in the individual.

2. The method according to claim 1, wherein the LSt cells are positioned in lamina XII and X in lumbar segments 3 (L3) and 4 (L4).

3. The method according to claim 2, wherein the LSt cells comprise neurotransmitters, neuropeptides, or combinations thereof.

4. The method according to claim 3, wherein the neuropeptides comprise galanin, cholecystokinin, enkephalin, glutamate, or combinations thereof.

5. The method according to claim 1, wherein the drug comprises an agonist of at least one neurotransmitter.

6. The method according to claim 1, wherein the drug comprises an antagonist of at least one neurotransmitter.

7. A method for manipulating ejaculatory reflex in an individual, comprising the step of administering to an individual a drug which interacts with lumbar spinothalamic (LSt) cells to manipulate ejaculatory reflex in the individual.

8. The method according to claim 7, wherein the LSt cells are positioned in lamina XII and X in lumbar segments 3 (L3) and 4 (L4).

9. The method according to claim 8, wherein the LSt cells comprise neurotransmitters, neuropeptides, or combinations thereof.

10. The method according to claim 9, wherein the neurotransmitters comprise gamma-amino-butyric-acid.

11. The method according to claim 9, wherein the neuropeptides comprise substance p, serotonin, somatostatin, galanin, vasointestinal peptide, calcitonin gene related peptide, or combinations thereof.

12. The method according to claim 7, wherein the drug comprises an agonist of at least one neurotransmitter.

13. The method according to claim 7, wherein the drug comprises an antagonist of at least one neurotransmitter.

14. A method for treating sexual dysfunction in an individual comprising the step of administering to the individual a drug which interacts with lumbar spinothalamic (LSt) cells to treat sexual dysfunction.

15. The method according to claim 14, wherein the LSt cells are positioned in lamina XII and X in lumbar segments 3 (L3) and 4 (L4).

16. The method according to claim 15, wherein the LSt cells comprise neurotransmitters, neuropeptides, or combinations thereof.

17. The method according to claim 16, wherein the neuropeptides comprise substance p, serotonin, somatostatin, galanin, cholecystokinin, enkephalin, glutamate, or combinations thereof, or combinations thereof.

18. The method according to claim 16, wherein the neurotransmitters comprise gamma-amino-butyric-acid.

19. The method according to claim 14, wherein the drug comprises an agonist of at least one neurotransmitter.

20. The method according to claim 14, wherein the drug comprises an antagonist of at least one neurotransmitter.

21. The method according to claim 14, wherein the sexual dysfunction comprises premature ejaculation, delayed ejaculation, control of ejaculatory function in an individual with spinal cord injury, anorgasmic ejaculation, or combinations thereof.
